(19) United States
(12) Patent Application Publication
(10) Pub. No.: US 2005/0171336 A1
(43) Pub. Date: Aug. 4, 2005

(54) Murine ortholog of the human disrupted-in-schizophrenia 1 gene

(76) Inventors: Jill A. Morris, Park Ridge, IL (US); Lei Ma, Eagleville, PA (US); Yuan Liu, Lansdale, PA (US)

Correspondence Address: MERCK AND CO., INC, P O BOX 2000, RAHWAY, NJ, 07065-0907, US

(21) Appl. No.: 10/514,510

(22) PCT Filed: May 20, 2003
(86) PCT No.: PCT/US03/15741

Related U.S. Application Data
(60) Provisional application No. 60/383,191, filed on May 24, 2002.

(51) Int. Cl. C12Q 1/68; A01K 67/027; C07H 21/04; C07K 16/28; C07K 14/705

(52) U.S. Cl. 530/350; 435/6; 435/69.1; 435/320.1; 435/354; 536/23.5; 800/18

(57) ABSTRACT

The present invention features Disc1 polypeptides, Disc1 nucleic acids, and recombinant Disc1 altered mice. The Disc1 amino acid sequence of SEQ ID NO: 1 and the nucleic acid sequence of SEQ ID NO: 2 provide the mouse ortholog to the human DISC1 amino acid sequence and nucleic acid sequence.

### CROSS-REFERENCE TO RELATED APPLICATIONS

The present application claims the benefit of U.S. Provisional Application No. 60/383,191, filed May 24, 2002, hereby incorporated by reference herein.

### BACKGROUND OF THE INVENTION

The references cited throughout the present application are not admitted to be prior art to the claimed invention.

Schizophrenia is a debilitating psychiatric disorder characterized by disordered thinking, hallucinations, and cognitive dysfunction. (Frances et al. ed. Diagnostic and Statistical Manual of Mental Disorders. Fourth Edition ed. 1994, American Psychiatric Association: Washington, D.C.) Family, twin and adoption studies have suggested that ˜50% of the risk of developing schizophrenia is genetic.

The human disrupted-in-schizophrenia 1 (DISC1) and the disrupted-in-schizophrenia 2 (DISC2) genes have been identified as genes that may play a role in susceptibility to psychiatric illness. (Millar et al. (2000) Hum. Mol. Genet., 9(9), 1415-1423.)

DISC1 and DISC2 genetic abnormalities have been associated with schizophrenia and related disorders. In a single Scottish family, the DISC1 open reading frame was found to be truncated by a balanced (1:11)(q42.1;q14.3) translocation. In this family, the translocation segregates not only with schizophrenia, but with other major mental illnesses, including schizoaffective disorder, bipolar disorder, and unipolar depression. The observed familial clustering of diseases is typical of sporadic schizophrenia. (Millar et al. (2000) Hum. Mol. Genet., 9(9), 1415-1423.)

Additional support for DISC1 playing a role in psychiatric illness comes from its chromosomal location. DISC1 was found to map next to the chromosomal marker D1S251, which localizes DISC1 to a region implicated in psychiatric illness. (Millar et al. (2001) Mol. Psychiatry, 6(2), 173-178.)

DISC1 is estimated to be 300 kb and contains 13 exons. (Millar et al. (2001) Mol. Psychiatry, 6(2), 173-178.) An identified open reading for DISC1 encodes a putative protein of 854 amino acids. (Millar et al. (2000) Hum Mol Genet, 9(9), 1415-1423.) The putative DISC1 protein contains an N-terminal region (amino acids 1-147) predicted to consist of one or more globular domains and a C-terminal region predicted to consist entirely of α-helix interspersed with several short loops. (Millar et al. (2000) Hum Mol Genet, 9(9), 1415-1423.)

DISC2 overlaps with DISC1 exon 9. (Millar et al. (2001) Mol Psychiatry, 6(2), 173-178.) DISC2 has been suggested to specify a non-coding RNA molecule that is antisense to DISC1. (Millar et al. (2000) Hum Mol Genet, 9(9), 1415-1423.)

### SUMMARY OF THE INVENTION

The present invention features Disc1 polypeptides, Disc1 nucleic acids, and recombinant Disc1 altered mice. The Disc1 amino acid sequence of SEQ ID NO: 1 and the nucleic acid sequence of SEQ ID NO: 2 provide the mouse ortholog to the human DISC1 amino acid sequence and nucleic acid sequence.

SEQ ID NO: 1 provides a reference sequence for Disc1 polypeptides. Disc1 polypeptides contain a region of at least 18 contiguous amino acids that is present in SEQ ID NO: 1. Disc1 polypeptides may contain additional regions beyond 18 contiguous amino acids present in SEQ ID NO: 1 and may contain amino acid regions not present in SEQ ID NO: 1.

SEQ ID NO: 2 provides a reference sequence for Disc1 nucleic acids. Disc1 nucleic acids contain a region that encodes a Disc1 polypeptide or contains at least 30 contiguous nucleotides that is present in SEQ. ID. NO. 2 or the complement thereof. Such Disc1 nucleic acids may contain additional regions present, or not present, in nucleic acid encoding for Disc1, or present in SEQ. ID. NO. 2 or the complement thereof.

Thus, a first aspect of the present invention describes a purified Disc1 polypeptide. The polypeptide comprises at least 18 contiguous amino acids of SEQ ID NO: 1.

A “purified polypeptide” represents at least 10% of the total protein present in a sample or preparation. In preferred embodiments, the purified polypeptide represents at least about 50%, at least about 75%, or at least about 95% of the total protein in a sample or preparation. Reference to “purified polypeptide” does not require that the polypeptide has undergone any purification and may include, for example, chemically synthesized polypeptide that has not been purified.

Another aspect of the present invention describes a recombinant nucleic acid that either:
a) encodes a Disc1 polypeptide and is transcriptionally coupled to an exogenous promoter;
b) is a Disc1 nucleotide sequence or the complement thereof and is attached to a solid support;
c) is provided by SEQ ID NO: 2;
d) is provided by a modified SEQ ID NO: 2 sequence; or
e) is provided by SEQ ID NO: 4.

A recombinant nucleic acid is a nucleic acid that contains two or more nucleic acid regions not naturally associated with each other and/or is present in a different environment than found in nature. Examples of recombinant nucleic acid includes nucleic acid containing a coding region and one or more regulatory elements not naturally associated with the coding region, exons joined together in DNA, expression vectors, and nucleic acid attached to a solid support. Recombinant nucleic acid containing recombined regions can be present inside a genome or may exist outside of the genome.

Another aspect of the present invention describes a recombinant cell comprising a nucleotide sequence encoding a Disc1 polypeptide that is transcriptionally coupled to an exogenous promoter. The exogenous promoter is a promoter not naturally associated with the nucleotide sequence. The cell contains an RNA polymerase that recognizes the promoter.

Another aspect of the present invention describes a recombinant cell made by a process comprising the step of introducing into a mouse cellular genome a recombinant nucleic acid encoding at least 18 contiguous bases of SEQ ID NO: 1.

Another aspect of the present invention features a purified antibody preparation comprising an antibody that selective binds to a polypeptide of SEQ ID NO: 1 over human DISC1 polypeptide (SEQ ID NO: 5). The antibody may also bind to fragments and/or variants of SEQ ID NO: 1.

A “purified antibody preparation” is a preparation where at least 10% of the antibodies present bind to a polypeptide of SEQ ID NO: 1. The preparation may contain polyclonal or monoclonal antibodies. In preferred embodiments, antibodies binding to Disc1 represent at least about 50%, at least about 75%, or at least about 95% of the total antibodies present. Reference to “purified antibody preparation” does not require that the antibodies in the preparation have undergone any purification.

Another aspect of the invention describes a recombinant Disc1 altered mouse. The mouse comprises an alteration in an allele encoding a Disc1 polypeptide comprising at least 20 contiguous amino acids of SEQ ID NO: 1, wherein the alteration substantially reduces, or increases, full length expression of Disc1 from the allele. The presence of nucleic acid encoding at least 20 contiguous amino acids of SEQ ID NO: 1 characterizes the nucleic acid as providing a Disc1 allele.

Another aspect of the present invention features a method for screening for a compound able to bind to a Disc1 polypeptide. The method involves the step of measuring the ability of the compound to bind to the polypeptide.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1. Human DISC1 (“oth:human”; SEQ ID NO: 5) and the murine ortholog (“oth:mouse”; SEQ ID NO: 1) were aligned by Clustal W alignment. There was 56% identity and 14% similarity (excludes identical amino acids) between the two proteins. An InterPro domain search revealed a weak suggestion of a tropomyosin (amino acids 349-366; and amino acids 556-581) and a bipartite nuclear localization signal (amino acids 331-348) in the human sequence (Apweiler et al. (2000) Bioinformatics, 16(12), 1145-1150). The mouse sequence had a low homology to DUF232 (amino acids 454-477). Arrow indicates translocation breakpoint. Bioinformatic analysis revealed three leucine zipper motifs conserved between mouse (amino acids 454-475; amino acids 461-482; and amino acids 603-624) and human (amino acids 458-479) (amino acids 465-486) (amino acids 607-628).

FIGS. 2A, 2B, and 2C. Comparison of human DISC1 (“oth:human”; SEQ ID NO: 6) and murine Disc1 nucleic acid (“oth:mouse”; SEQ ID NO: 2).

FIG. 3. Mouse Disc1 splice variant amino acid sequence (SEQ ID NO: 3).

FIG. 4. Mouse Disc1 splice variant encoding nucleic acid sequence along with a TGA stop codon (SEQ ID NO: 4).

FIG. 5. A BAC map of the Disc1 genomic region. Two BACs were identified using the TIOR BAC end sequencing database. (Zhao et al. (2001) Genome Res, 11(10), 1736-1745.) 418L11 contains sequences from 946-1446 of the Tsnax gene. 236F19 contains nucleotides 1500-2410 of the Tsnax. Bac259E12 was identified by hybridization of a Disc1 probe (nucleotides 2376-2490) against a mouse BAC library (Incyte).

### DETAILED DESCRIPTION OF THE INVENTION

Disc1, the mouse ortholog to human DISC1, has been identified and cloned. Human DISC1 translocation has been associated with psychiatric diseases such as schizophrenia, schizoaffective disorder, bipolar disorder, and unipolar depression.

The present invention include Disc1 polypeptides and nucleic acids. Disc1 polypeptides and nucleic acids have a variety of different uses such as providing research tools for studying Disc1 polypeptide function and expression in a cell; studying the involvement of Disc1 with psychiatric diseases; identifying Disc1 nucleotide polymorphism(s); and creating recombinant Disc1 deficient mice.

A recombinant Disc1 deficient mouse can be used, for example, as model to examine the involvement of Disc1 with psychiatric diseases, and the ability of compounds to compensate for the effect of a Disc1 alteration.

I. Disc1 Polypeptides

Disc1 polypeptides contain a region of at least 18 contiguous amino acids that is present in SEQ ID NO: 1. Disc1 polypeptides have a variety of uses, such as being used as an immunogen to produce antibodies binding to Disc1 and being used as a target to identify compounds binding to the Disc1.

The presence of at least 18 contiguous amino acids of SEQ ID NO: 1 provides a unique structural tag for a Disc1 polypeptide and a sufficient polypeptide region to achieve a useful purpose. The at least 18 contiguous amino acids can, for example, provide an immunogen to generate an antibody. In different embodiments the Disc1 polypeptide contains a tag of at least 20 contiguous amino acids of SEQ ID NO: 1; at least 40 contiguous amino acids of SEQ ID NO: 1, at least 80 contiguous amino acids of SEQ ID NO: 1; or comprises or consists of SEQ ID NO: 1.

Disc1 polypeptides may contain additional SEQ ID NO: 1 regions in addition to a Disc1 tag and may contain amino acid regions not present in SEQ ID NO: 1. Disc1 polypeptides include full length Disc1 of SEQ ID NO: 1, variants of SEQ ID NO: 1 containing a Disc1 tag, and chimeric polypeptides containing a Disc1 polypeptide and amino acid region(s) not from SEQ ID NO: 1.

Variants of SEQ ID NO: 1 containing a Disc1 tag include naturally occurring variants such as splice variants and/or polymorphic variants. SEQ ID NO: 3 provides the sequence of a splice variant that has an amino acid alteration. Examples of SEQ ID NO: 1 variants are also provided in Example 2, Table 3, infra. The variants provided in Table 3 were obtained from a splice variant and different PCR product reactions.

In additional embodiments concerning Disc1 polypeptide variants, SEQ ID NO 1: is modified with one or more of the following modifications:
amino acid 46: A to V;
amino acid 58: G to D;
amino acid 111: E to D;
amino acid 214: F to L; and
amino acid 231: C to R.
   Preferred combinations of modifications correspond to those found in a particular PCR product (amino acids 46, 58, 111 and 201 were from one PCR product; amino acid 214 was from one PCR product; and amino acids 231 and 397 was from a splice variant).

Chimeric polypeptides containing a Disc1 tag can contain non-Disc1 regions chosen to achieve a particular purpose or to produce a polypeptide that can substitute for Disc1 or a fragment thereof. Particular purposes that can be achieved using appropriate non-Disc1 regions include providing a marker for isolation and enhancing an immune response.

In additional embodiments, the Disc1 polypeptide contains at least 18, at least 20, at least 40 or at least 80 contiguous amino acids where the encoding nucleic acid spans two or more exons. The amount of contiguous amino acids corresponding to a particular exon can vary. In different embodiments the Disc1 polypeptide contains at least 9, at least 10, at least 20, or at least 40 amino acids contiguous amino acids corresponding to two or more different exons.

The amino acids sequences in SEQ ID NO: 1 encoded by different exons are assigned as follows:

Exon 1
MQGGGPRDAPIHSPSHGA
Exon 2
SGHGLPPAVAPQRRRLTRRPGYMRSTAGSGIGFLSPAVGMPHPSSAGLTG
QQSQHSQSKAGQCGLDPGSHCQASLVGKPFLKSSLVPAVASEGHLHPAQR
SMRKRPVHFGVHSKNDSRQSEKLTGSFKPGDSGCWQELLSSDSFKSLAPS
LDAPWNTGSRGLKTVKPLASSALNGPADIPSLPGFQDTFTSSFSFIQLSL
GAAGERGEAEGCLPSREAEPLHQRPQEMAAEASSSDRPHGDPRHLWTFSL
HAAPGLADLAQVTRSSSRQPECGTVSSSSDTVFSSQDASSAGGRGDQGGG
WADAHGWHTLLREWEPMLQDYLLSNRRQLE
Exon 3
VTSLILKLQKCQEKAVEDGDYDT
Exon 4
ETLRQRLEELEQEKGHLSWALPSQQPALRSFLGYLAAQIQVALHGATQ
Exon 5
AGSDDPEAPLEGQLRTTAQDSLPASITRRDWLIREKQQLQ
Exon 6
KEIEALQARMSALEAKEKRLSQELEEQEVLLRWPGCDLMALVAQMSPGQL
QEVSKALGETLTSANQAPFHVEPPETLR
Exon 7
LRERTKSLNLAVRELTAQ
Exon 8
VCSGEKLCSSLRRRLSDLDTRLPALLEAKMLALS
Exon 9
SCFSTAKELTEEIWALSSEREGLEMFLGRLLALSSRNSRRLGILKEDYLR
CRQDLALQDAAH
Exon 10
TRMKANTVKCMEVLEGQLS
Exon 11
CRCPLLGRVWKADLETCQLLMQSLQLQEAGSSPHAEDEEQVHSTGEAAQT
AALAVPRTPHPEEEKSPLQVLQEWDTHSALSPHCAAGPWKE
Exon 12
DSHIVSAEVGEKCEAIGVRLLHLEDQLLGAMYSHDEALF
Exon 13
SLQGELQTVKETLQAMILQLQPTKEAGEASASYPTAGAQETEA

Polypeptides can be produced using standard techniques including those involving chemical synthesis and those involving biochemical synthesis. Techniques for chemical synthesis of polypeptides are well known in the art. (See e.g., Vincent, in Peptide and Protein Drug Delivery, New York, N.Y., Decker, 1990.)

Biochemical synthesis techniques for polypeptides are also well known in the art. Such techniques employ a nucleic acid template for polypeptide synthesis. The genetic code providing the sequences of nucleic acid triplets coding for particular amino acids is well known in the art. (See, e.g., Lewis GENES IV, p. 119, Oxford University Press, 1990.) Examples of techniques for introducing nucleic acid into a cell and expressing the nucleic acid to produce protein are provided in references such as Ausubel, Current Protocols in Molecular Biology, John Wiley, 1987-1998, and Sambrook et al., Molecular Cloning, A Laboratory Manual, 2^{nd} Edition, Cold Spring Harbor Laboratory Press, 1989.

II. Disc1 Antibodies

Antibodies recognizing Disc1 can be produced using a polypeptide containing SEQ ID NO: 1 or a fragment thereof as an immunogen. Antibodies recognizing Disc1 have different uses such as being used to identify the presence of Disc1, to isolate Disc1 polypeptides, and to study Disc1 expression.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; Kohler, et al., Nature 256:495-497, 1975; and Schweitzer et al. Current Opinion in Biotechnology 13:14-19, 2002.

III. Binding Assay

Disc1 polypeptides can be used in binding studies to identify compounds binding to the receptor. Preferably, binding studies are performed using Disc1 expressed from a recombinant nucleic acid. More preferably, recombinantly expressed Disc1 consists of the SEQ. ID. NO. 1, SEQ. ID. NO. 3, or a modified SEQ. ID. NO. 1 containing one or more modifications selected from the group consisting of:
amino acid 46: A to V;
amino acid 58: G to D;
amino acid 111: E to D;
amino acid 214: F to L; and
amino acid 231: C to R.

Binding assays can be performed using individual compounds or preparations containing different numbers of compounds. A preparation containing different numbers of compounds having the ability to bind to a Disc1 polypeptide can be divided into smaller groups of compounds that can be tested to identify the compound(s) binding to the Disc1 polypeptide.

Binding assays can be performed using Disc1 present in different environments. Such environments include, for example, cell extracts and purified cell extracts containing a Disc1 recombinant nucleic acid; and also include, for example, the use of a purified Disc1 polypeptide produced by recombinant means which is introduced into a different environment.

IV. Disc1 Nucleic Acid

Disc1 nucleic acid contains a region encoding a Disc1 polypeptide or contains at least 30 contiguous nucleotides present in SEQ ID NO: 2 or the complement thereof. Disc1 nucleic acids have a variety of uses, such as being used as a hybridization probe or polymerase chain reaction (PCR) primer to identify the presence of Disc1 variants and orthologs; being used as a hybridization probe to monitor Disc1 expression; being used as an antisense nucleic acid to examine Disc1 functions; being used for recombinant expression of Disc1 polypeptides; and/or being used in the construction of recombinant mice having an altered Disc1 allele.

The presence of a region that encodes a Disc1 polypeptide or contains at least 30 contiguous nucleotides that is present in SEQ ID NO: 2 or the complement thereof provides a unique structural tag and a sufficient nucleic acid region to achieve a useful purpose. Examples of particular purposes include providing a sequence that encodes a Disc1 polypeptide and/or providing a sequence that can selectively hybridize to Disc1 MRNA under appropriate stringency conditions. Selective hybridization indicates that the nucleic acid region can preferentially hybridize to murine Disc1 MRNA over at least human DISC1 MRNA.

Disc1 nucleic acid may contain regions in addition to a region that provides the Disc1 tag. Additional regions include Disc1 related regions such as additional regions encoding for SEQ ID NO: 1 polypeptides or variants thereof, additional SEQ ID NO: 3 regions or variants thereof, additional regions complementary to SEQ ID NO: 3 and variants thereof; and non-Disc1 related regions.

Non-Disc1 related regions are preferably chosen to achieve a particular purpose. Examples of non-Disc1 related regions that can be used to achieve a particular purpose include capture regions that can be used as part of a sandwich assay, reporter regions that can be probed to indicate the presence of the nucleic acid, expression vector regions, and regions encoding for immune enhancing polypeptides. Variants of SEQ ID NO: 1 are described above in Section I.

Variants of SEQ ID NO: 2 contain a Disc1 tag and include naturally occurring variants such as splice variants and/or polymorph variants of SEQ ID NO: 2. SEQ ID NO: 4 provides the sequence of a splice variant. Examples of SEQ ID NO: 2 variants are also provided in Example 2, Table 3, infra. The variants provided in Table 2 were obtained from a splice variant and different PCR product reactions.

In additional embodiments concerning Disc1 nucleic acid variants, SEQ ID NO 2: is modified with one or more of the following modifications:
nucleotide 137: C to T;
nucleotide 173: G to A;
nucleotide 333: G to T;
nucleotide 606: C to T;
nucleotide 640: T to C;
nucleotide 691: T to C; and
nucleotide 1191: G to A.
   Preferred combinations of modifications correspond to those found in a particular PCR product (nucleotides 137, 173, 333 and 606 were from one PCR product; nucleotide 640 was from one PCR product; and nucleotides 691 and 1191 was from the splice variant).

In additional embodiments the Disc1 nucleic acid contains at least 30, at least 60, or at least 90 contiguous nucleotides, where the nucleotides either encode amino acids spanning at least two exons, are present in two or more exons, or are complementary to nucleotides present in two or more exons. The amount of nucleic acid corresponding to a particular exon can vary. In different embodiments the Disc1 nucleic acid encodes a polypeptide containing at least 9, at least 10, at least 20, or at least 40 contiguous amino acids from two or more different exons; and the Disc1 nucleic acid contains at least 15, at least 30, or at least 45 contiguous bases from two or more different exons, or the complement thereof.

Table 1 illustrates the intron/exon boundaries and genomic structure of the Disc1 gene.

TABLE 1

| | | | | | | |
|---|---|---|---|---|---|---|
| | | | Intron | | | |
| | Exon | Position | Size | | Exon Boundary | |
| Exon | Size bp^{a} | In message^{b} | in bp^{a} | Splice Acceptor Site | Sequence | Splice Donor Site |

| | | | | | | |
|---|---|---|---|---|---|---|
| 1 | | 1-90 | >48,000 | | .........GCGCAG | gtagggcccggggttctggaggagg |
| | | | | | SEQ ID NO: 21 | |
| 2 | 992 | 91-1082 | >1.9 | acactgttttctcttctcttctcag | ACAGTG...CTGGAG | gtgtgtgtgcttctggaatcgggtc |
| | | | | SEQ ID NO: 7 | | SEQ ID NO: 22 |
| 3 | 70 | 1083-1152 | 34,050 | atgtttccctttctcacccacacag | GTCACT...ATACTG | gtgagtccaaagctgttcgtagaca |
| | | | | SEQ ID NO: 8 | | SEQ ID NO: 23 |
| 4 | 148 | 1153-1300 | 7,422 | tgcttttacctctttgggtttccag | CAGAGA...CCAAAG | gtgagtacccgtggatgccaccaca |
| | | | | SEQ ID NO: 9 | | SEQ ID NO: 24 |
| 5 | 121 | 1301-1421 | 3,087 | accaatgcatgtctgttacttgaag | GGCCGG...TTGCAG | gtgagtggaatagaatcttccagaa |
| | | | | SEQ ID NO: 10 | | SEQ ID NO: 25 |
| 6 | 236 | 1422-1657 | >12,900 | atctgttccccctctctctctgcag | AAGGAA...CAGGAG | gtactggtgactttctgagtttcca |
| | | | | SEQ ID NO: 11 | | SEQ ID NO: 26 |
| 7 | 55 | 1658-1712 | 6541 | caatgctcctttctaatttctctag | CCTCCG...GCTCAG | gtaagcccaccctcctcccattttc |
| | | | | SEQ ID NO: 12 | | SEQ ID NO: 27 |
| 8 | 103 | 1713-1815 | >9400 | ttgattctgccgtttctcctggcag | GTGTGC...TATCAG | gtaactgcagaggcacttatattca |
| | | | | SEQ ID NO: 13 | | SEQ ID NO: 28 |
| 9 | 189 | 1816-2004 | >51,900 | tcctctctcccccactgtgttgcag | GAAGCT...CCCACA | gtgagtagcccccagccaaagcctc |
| | | | | SEQ ID NO: 14 | | SEQ ID NO: 29 |
| 10 | 61 | 2005-2065 | 14,626 | tgctcacgttgggtttttcttgcag | AAACAC...GAGCAG | gtaagttgtgtgtgtgtgtgggggg |
| | | | | SEQ ID NO: 15 | | SEQ ID NO: 30 |
| 11 | 274 | 2066-2339 | 17,890 | ccatgcctgccttcctctgtcgtag | CTGCAG...AAAGAG | gtttgtcctgtgtgtatggctttgt |
| | | | | SEQ ID NO: 16 | | SEQ ID NO: 31 |
| 12 | 118 | 2340-2457 | 9949 | gacacatctctcattctctgaccag | GATTCT...TCTTTC | atatccttttcagtctctcgggaat |
| | | | | SEQ ID NO: 17 | | SEQ ID NO: 32 |
| 13 | 137 | 2458-2594 | 444 | ttgtgtgctccttaacaatgtctac | AGTCTC...TGAGGT | gtgagtgtggagggggacgggggag |
| | | | | SEQ ID NO: 18 | | SEQ ID NO: 33 |
| 14 | 263 | 2595-2857 | 24 | ttttctttctttctttttccttcag | CCTGCT...TGCTGC | tgtcgccgccgccaccaccaccac |
| | | | | SEQ ID NO: 19 | | SEQ ID NO: 34 |
| 15 | 302 | 2858-3159 | | tgtcgccgccgccaccaccaccac | CACCAC... | |
| | | | | SEQ ID NO: 20 | | |

^{a}base pair(s)
^{b}The nucleotide position of the exons in the Disc1 message are indicated with the A of ATG being +1.

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989.

Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. Amino acids are encoded by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., Molecular Cloning, A Laboratory Manual, 2^{nd} Edition, Cold Spring Harbor Laboratory Press, 1989, and Kacian et al., U.S. Pat. No. 5,480,784.

V. Obtaining Additional Nucleic Acid Related To Disc1

The guidance provided herein can be used to obtain nucleic acid sequences encoding Disc1 related polypeptides from different sources. Obtaining such nucleic acids is facilitated using probes and primers and by the proper selection of hybridization conditions.

Probes and primers can be designed based on Disc1 nucleic acid and amino acid sequences. Adjusting hybridization conditions is useful for controlling probe or primer specificity.

Techniques employed for hybridization detection and PCR cloning are well known in the art. Nucleic acid detection techniques are described, for example, in Sambrook et al., Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

Disc1 probes and primers can be used to screen nucleic acid libraries containing, for example, genomic DNA or cDNA. Such libraries are commercially available, and can be produced using techniques such as those described in Ausubel, Current Protocols in Molecular Biology, John Wiley, 1987-1998.

VI. Disc1 Probes

Disc1 probes contain a region that can specifically hybridize to Disc1 target nucleic acid under appropriate hybridization conditions and can distinguish Disc1 nucleic acid from non-target nucleic acids. Probes for Disc1 can also contain nucleic acid that are not complementary to Disc1 nucleic acid.

Probes can be free in solution or attached to a solid support. Probes covalently or non-covalently attached to a solid support can be used, for example, to monitor expression of different genes. Probes can be attached to a solid support through different techniques such as spotting synthesized probe onto a support or synthesizing probes in a stepwise fashion onto a support. Techniques for monitoring gene expression can be found in references such as U.S. Pat. No. 5,965,352 and U.S. Pat. No. 6,203,987.

Probes are composed of nucleic acids or derivatives thereof such as modified nucleic acid and peptide nucleic acid. Modified nucleic acid includes nucleic acid with one or more altered sugar groups, altered internucleotide linkages, and/or altered nucleotide purine or pyrimidine bases. References describing modified nucleic acid include WO 98/02582, U.S. Pat. No. 5,859,221 and U.S. Pat. No. 5,852,188, each of which are hereby incorporated by reference herein.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the Tm of the produced hybrid. The higher the Tm the stronger the interactions and the more stable the hybrid. Tm is affected by different factors well known in the art such as the degree of complementarity, the type of complementary bases present (e.g., A-T hybridization versus G-C hybridization), the presence of modified nucleic acid, and solution components. (E.g., Sambrook et al., Molecular Cloning, A Laboratory Manual, 2^{nd} Edition, Cold Spring Harbor Laboratory Press, 1989.)

Stable hybrids are formed when the Tm of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

VII. Recombinant Expression

Disc1 polypeptides can be expressed from recombinant nucleic acid in a suitable host, or in a test tube using a translation system. Preferably, expression is achieved in a host cell using an expression vector.

An expression vector contains recombinant nucleic acid that includes a region encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Expression vectors providing suitable levels of polypeptide expression in different hosts are well known in the art. Mammalian expression vectors well known in the art include pcDNA3 (Invitrogen), pMC1neo (Stratagene), pXT1 (Stratagene), pSG5 (Stratagene), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), pCI-neo (Promega) and .lambda.ZD35 (ATCC 37565). Bacterial expression vectors well known in the art include pET11a (Novagen), lambda gt11 (Invitrogen), pcDNAII (Invitrogen), and pKK223-3 (Pharmacia). Fungal cell expression vectors well known in the art include pYES2 (Invitrogen) and Pichia expression vector (Invitrogen). Insect cell expression vectors well known in the art include Blue Bac III (Invitrogen).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK.sup.-) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

To enhance expression in a particular host it may be useful to modify a particular encoding sequence to take into account codon usage of the host. Codon usage of different organisms are well known in the art. (See, Ausubel, Current Protocols in Molecular Biology, John Wiley, 1987-1998, Supplement 33 Appendix 1C.)

Expression vectors may be introduced into host cells using standard techniques. Examples of such techniques include transformation, transfection, lipofection, protoplast fusion, and electroporation.

Nucleic acid encoding a polypeptide can be expressed in a cell without the use of an expression vector. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection.

VIII. Production of Disc1 Deficient and Transgenic Mice

Based on the guidance provided herein, different types of mice which are deficient in Disc1, or overexpress wild type, truncated, or otherwise mutant Disc1 (referred to as knockout, transgenic, or knock-in mice), can be produced. Such mice may mimic the truncation present in human schizophrenics with DISC1 truncation reported by Millar et al. (2000), thus producing a mouse model for aspects of the human schizophrenic phenotype or schizophrenia as a whole. A scheme for producing Disc1 deficient mice involves producing male and female mice with an altered Disc1 allele and breeding the mice to produce mice having alterations in both alleles.

Techniques for producing mice with an altered genome are well known in the art. (Ausubel, Chapter 23, Manipulating the Mouse Genome, Current Protocols in Molecular Biology, John Wiley, 2001). An example of a scheme for producing a mouse with an altered Disc1 allele involves the following:
(a) altering the Disc1 allele in a mouse embryonic stem cell by homologous recombination with a transgene to produce an altered embryonic stem cell;
(b) introducing the altered embryonic stem cell into a mouse blastocyst to produce an altered blastocyst;
(c) introducing the altered blastocyst into a pseudopregnant mouse to produce a pregnant mouse;
(d) allowing the pregnant mouse to produce offspring; and
(e) screening the offspring for the presence of an altered Disc1 allele to identify a Disc1 deficient mouse.

Genetic elements involved in gene expression include transcription and translation elements such as a promoter, splicing sites, polyadenylation region, and ribosome binding site. Removing or altering these elements will alter the production of Disc1 protein from the Disc1 gene.

Disc1 structural gene alterations can be used to substantially reduce or eliminate full-length expression of the polypeptide from the allele. Preferred alterations to the Disc1 structural gene involve either knocking out the gene or producing a gene that encodes bases 1-593 corresponding to the amino region up to the translocation break point.

A deletion in a Disc1 allele can be accompanied by an insertion of additional nucleic acid. Additional nucleic acid that may be inserted includes nucleic acid encoding a selectable marker having an independent promoter and nucleic acid encoding a reporter protein transcriptionally coupled to the Disc1 promoter. Examples of reporter protein that can be used in chimeric mice are β-galactosidase (lacZ) and green fluorescent protein (GFP) and its derivatives.

Initial alterations are preferably produced using a transgene containing one or more selectable makers and nucleic acid targeting Disc1 for insertion by homologous recombination. Homologous recombination can be performed to create alterations in Disc1 and/or remove Disc1 regions. Markers can be used to facilitate screening for the insertion into a mouse genome, and for the insertion occurring by homologous recombination. (Ausubel, Chapter 23, Manipulating the Mouse Genome, Current Protocols in Molecular Biology, John Wiley, 2001.)

A transgene used for homologous recombination may contain recombinase systems, which may be employed to excise inserted nucleic acid. Examples of recombinase systems include the bacteriophage recombinase Cre/loxP system and the yeast recombinase Flp/FRT system. (Ausubel, Chapter 23, Manipulating the Mouse Genome, Current Protocols in Molecular Biology, John Wiley, 2001, and U.S. Pat. No. 5,564,182.) loxP recognition sites can be positioned 3′ and 5′ of a region to be removed and excised by Cre recombinase. Similarly, frt recognition sites can be positioned 3′ and 5′ of a region to be removed and excised by Flp recombinase.

Screening for mice containing an altered Disc1 allele can be achieved using techniques such as those measuring the production of Disc1 mRNA transcripts and whether any produced Disc1 transcript is different from wild-type transcript. Techniques for measuring Disc1 MRNA transcripts and the type of transcript include nucleic acid hybridization analysis such as a Southern analysis that can detect the production and size of transcripts, and the use of smaller nucleic acid probes specific for a particular sequence. PCR can also be employed to measure Disc1 mRNA transcripts. Western blotting and immunohistochemistry can also be used to detect any full length or partial Disc1 protein in these animals.

### EXAMPLES

Examples are provided below to further illustrate different features of the present invention. The examples also illustrate useful methodology for practicing the invention. These examples do not limit the claimed invention.

### Example 1

### Materials and Methods

This example describes different materials and methods that were employed to clone and study Disc1.

Genomic Identification

Bioinformatic analysis of the draft mouse genomic sequence identified four mouse genomic sequences with homology to the human DISC1. The mouse sequences were identified by searching public mouse genomic shotgun sequences employing Blast (Altschul et al., (1997) Nucleic Acids Res, 25(17), 3389-402).

cDNA Cloning

Primers were designed based on mouse genomic sequences. A 1779 bp and a 1590 bp product were obtained by PCR using either mouse heart or brain Marathon-Ready cDNA (Clontech) as template and primers TTCATCCAACTCTCCCTTGG (SEQ ID NO: 35) and GAGAGCTTCGTCGTGACTG (SEQ ID NO: 36). PCR was carried out using Pfu Turbo DNA polymerase (Stratagene). Each 50 μl reaction contained 2.5 U of enzyme, 0.2 μM of each primer, 0.2 mM of each dNTP, 10 mM KCl, 10 mM (NH₄)₂SO₄, 20 mM Tris-Cl (pH 8.75), 20 mM MgSO₄, 0.1% Triton X-100, 0.1 mg/ml BSA and 2% DMSO. The reaction utilized 35 cycles with a denaturation step of 20 seconds at 94° C., an annealing step of 1 minute at 60° C., and a synthesis step of 3 minutes at 72° C. The PCR products have been cloned into PCR-Blunt II-TOPO vector (Invitrogen) using standard methods and sequenced.

5′RACE (Rapid Amplification of cDNA Ends) products were obtained using the Pfu Turbo DNA polymerase and the same reaction buffer described above. The PCR amplification was done with 32 cycles with a denaturation step of 20 seconds at 94° C., an annealing and synthesis step of 3 minutes at 68° C., with mouse heart brain Marathon-Ready cDNA (Clontech) as template (gene-specific primer CATTCTGGTTGCCTGCTGCTGC) (SEQ ID NO: 37). It was followed by a nested PCR reaction using primer ACCTGAGCCAAGTCTGCCAAGC (SEQ ID NO: 38) with 25 amplification cycles. The PCR products have been cloned into PCR-Blunt II-TOPO vector (Invitrogen) using standard methods and sequenced.

3′RACE products were obtained using the Pfu Turbo DNA polymerase and same reaction buffer above except excluding DMSO. PCR amplification was run using 32 cycles with a denaturation step of 20 seconds at 94° C., an annealing and synthesis step of 3 minutes at 68° C., with mouse heart brain Marathon-Ready cDNA (Clontech) as template (gene-specific primer CTGCTGAAGTTGGAGAAAAGTGCG) (SEQ ID NO: 39). It was followed by a nested PCR reaction using primers GGCCATGTACAGTCACGACGAAG (SEQ ID NO: 40) or GAGCTCCAGACGGTGAAGGAAAC (SEQ ID NO: 41) with 25 cycles. The PCR products have been cloned into PCR-Blunt II-TOPO vector (Invitrogen) using standard methods and sequenced.

Genomic Structure

A 3′ mouse Disc1 cDNA sequence (nucleotides 2376-2490) was used as a probe to screen a BAC (Bacterial Artificial Chromosome) Mouse library (Incyte Genomics). Standard procedures were used for hybridization as recommended by the manufacturer. Double-stranded probe was labeled with [α-³²P]dCTP using rediprimer II (Amersham Pharmacia Biotech rediprimer II random prime labeling system) and purified using Princeton Separations Centri-Sep columns.

The positive BAC clone was confirmed by PCR (primer set 1, GGATTCTCACATCGTTTCTGC (SEQ ID NO: 42) and GAGAGCTTCGTCGTGACTG (SEQ ID NO: 43); primer set 2, GAAATGGCCACTATACCTGC (SEQ ID NO: 44) and CGGCAGCAGTGGTTGTGA) (SEQ ID NO: 45). PCR was carried out using AmpliTaq Gold DNA polymerase (Applied Biosystems). Each 50 μl reaction contained 1.25 U of enzyme, 0.2 μM of each primer, 0.2 mM of each dNTP, 10 mM Tris-Cl (pH 8.3), 50 mM KCl, 1.5 mM MgCl₂, 0.001% (w/v)gelatin. Following 9 minutes incubation at 94° C., the reaction utilized 32 cycles with a denaturation step of 20 seconds at 94° C., an annealing step of 30 seconds at 60° C., and a synthesis step of 1 minute at 72° C.

The TRAX gene is located upstream from DISC1 on human chromosome 1q42. PCR results showed one of the mouse BAC clone positive for 3′ mouse TRAX is also positive for 5′ mouse Disc1 by PCR. Primers CCACATGCTTTCAACGAGTT (SEQ ID NO: 46) and AGAGCAGGTACCAGGACTGAC (SEQ ID NO: 47) were used for Tsnax. Two Disc1 primer sets were used (set 1, TTCATCCAACTCTCCCTTGG (SEQ ID NO: 48) and GGGCCTGTCTGAGCTAGATG (SEQ ID NO: 49); set 2, AGACTTGGCTCAGGTGACGA (SEQ ID NO: 50) and GCGGTTGCTCAGTAGGTAG) (SEQ ID NO: 51). PCR conditions were as same as above.

Northern Blot Analysis

Clontech mouse multiple tissue were probed with Disc1 (nucleotides 2376-2490). The probe was obtained by PCR using mouse heart Marathon-Ready cDNA (Clontech) as template. Disc1 is weakly present as transcripts of ˜7 kb and ˜4.4 kb in heart, brain, kidney and testis.

Low stringency hybridization was carried out on Clontech rat multiple tissue northern blots at 60° C. A probe corresponding to nucleotides 1138-2497 of Disc1 was obtained by excising one of the Disc1 heart cDNA clones using HindIII and EcoR1. A ˜7 kb transcript showed in heart, and skeletal muscle, and another ˜1.35 kb transcript showed in heart, liver, kidney and brain. Expression level was higher in heart and liver than in skeletal muscle and brain.

Bioinformatic Analysis

Two BACs were identified from the TIGR BAC end sequencing project by submitting murine TRAX cDNA sequence to the database www.tigr.org. (Zhao et al. (2001) Genome Res, 11(10), 1736-1745.)

Human DISC1 and mouse Disc1 DNA and protein sequences were aligned using a Clustal W program. (Thompson et al., (1994) Nucleic Acids Res, 22(22), 4673-80.) The human and murine sequences were characterized for subsequences using PROSITE. (Bairoch, (1991) Nucleic Acids Res, 19(Suppl), 2241-2245, Henikoff et al., (1991) Nucleic Acids Res, 19(23), 6565-6572.) Human and murine DISC1 sequences were both positive for leucine zipper motifs. Homologies to DUF232, tropomyosin and bipartite nuclear localization signal were found by searching the murine or human sequence using the InterPro program. (Apweiler et al. (2000) Bioinformatics, 16(12), 1145-1150.)

In Situ Hybridization

C57BL6 male mice (20-25 g; Taconic; Germantown, N.Y.) were housed in the animal care facility (AAALAC certified) with a 12-hour light, 12-hour dark photoperiod and free access to tap water and rodent chow. After acclimation (5-10 days), the animals were euthanized with an overdose of CO₂, their brains frozen and 20 μm coronal cryostat sections collected on gelatin-coated slides.

A fragment (bases 1138-2497) of the mouse Disc1 was excised from a heart cDNA clone with HindIII and EcoRI and subcloned into a pBluscript vector (Stratagene, La Jolla, Calif.). The plasmid was then used to generate ³⁵S-UTP labeled cRNA probes for in situ hybridization.

Briefly, the section-mounted slides were postfixed in 4% paraformaldehyde, treated with acetic anhydride and then delipidated and dehydrated with chloroform and ethanol. The slides were then hybridized with 200 μl (6×10⁶ DPM/slide) of an antisense or sense (control) riboprobe for Disc1 mRNA in a 50% formamide hybridization mix and incubated overnight at 55° C. in a humidified slide chamber without coverslipping. In the morning, the slides were washed in 2×SSC/10 mM DTT, treated with RNase A (20 μg/ml) and washed in 67° C. in 0.1×SSC to remove nonspecific label. After dehydration, the slides were opposed to BioMax (BMR-1; Kodak) x-ray film for 3 days and then dipped in NTB2 nuclear emulsion. The slides were exposed for 4-6 weeks, photographically processed, stained in cresyl violet and cover-slipped.

### Example 2

### Cloning of Disc1

Searching the DISC1 protein sequence against the public mouse genomic database (http://www.ncbi.nlm.nih.gov/genome/seq/MmHome.html) identified four mouse genomic DNA sequences corresponding to DISC1 sequences (Table 2). These sequences corresponded to exons 2, 6, 12 and 13 of the human DISC1 genomic sequence. (Millar et al. (2001) Mol Psychiatry, 6(2), 173-178.) Primers against mouse genomic fragments 1 and 4 were used to PCR amplify the central portion of the Disc1 from both whole brain and heart cDNA libraries. 5′ and 3′ RACE were then used to obtain the rest of the orthologous Disc1 sequence.

TABLE 2

| | Amino Acid in | | Amino Acid in |
|---|---|---|---|
| | Human | Human Exon | Mouse |
| Genomic | 123-322 | Exon 2 | 133-322 |
| sequence 1 | | | |
| Genomic | 465-544 | Exon 6 | 461-540 |
| sequence 2 | | | |
| Genomic | 769-807 | Exon 12 | 768-806 |
| sequence 3 | | | |
| Genomic | 808-842 | Exon 13 | 807-840 |
| sequence 4 | | | |

The Disc1 cDNA is 3190 bp in length, with an open reading frame of 2553 bp, corresponding to a protein 851 amino acids in length. An in-frame splice variant was also identified (SEQ ID NOs: 3 and 4).

The splice variant is 3001 bp in length, with 189 base pairs deleted compared to the full-length mouse cDNA. With nucleotide +1 being from the ATG, nucleotides +1843 to +2031 are spliced out in this variant; it has an open reading of 2364 bp, corresponding to a protein 788 amino acids in length.

A splice variant of human DISC1 was previously identified. (Millar et al. (2000) Hum. Mol. Genet, 9(9), 1415-23.) However, it is in a different location in the gene than the Disc1 splice variant. Both the full-length Disc1 sequence and the splice variant sequence were amplified in the brain and the heart cDNA libraries.

Multiple single nucleotide polymorphisms (SNPs) were also identified during the cloning of Disc1 (Table 3)

TABLE 3
Single Nucleotide Polymorphisms

| Position | Nucleotide | |
|---|---|---|
| (A in ATG is +1) | Change | Amino Acid |
| 137 | C → T | A → V |
| 173 | G → A | G → D |
| 333 | G → T | E → D |
| 606 | C → T | P → P |
| 640 | T → C | F → L |
| 691* | T → C | C → R |
| 1191* | G → A | Q → Q |

*polymorphism found in splice variant
The polymorphisms at positions 137, 173, 333, 606 are from the same PCR product and the polymorphism at position 640 is from a different PCR product.

### Example 3

### Bioinformatic Analysis

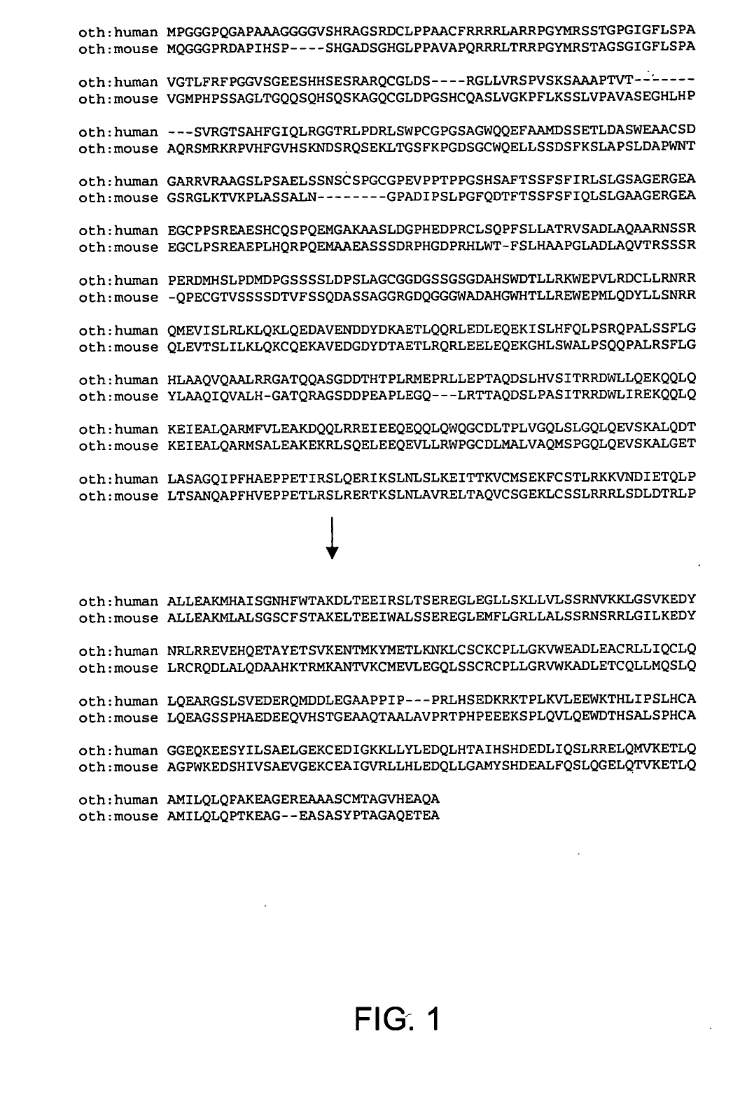

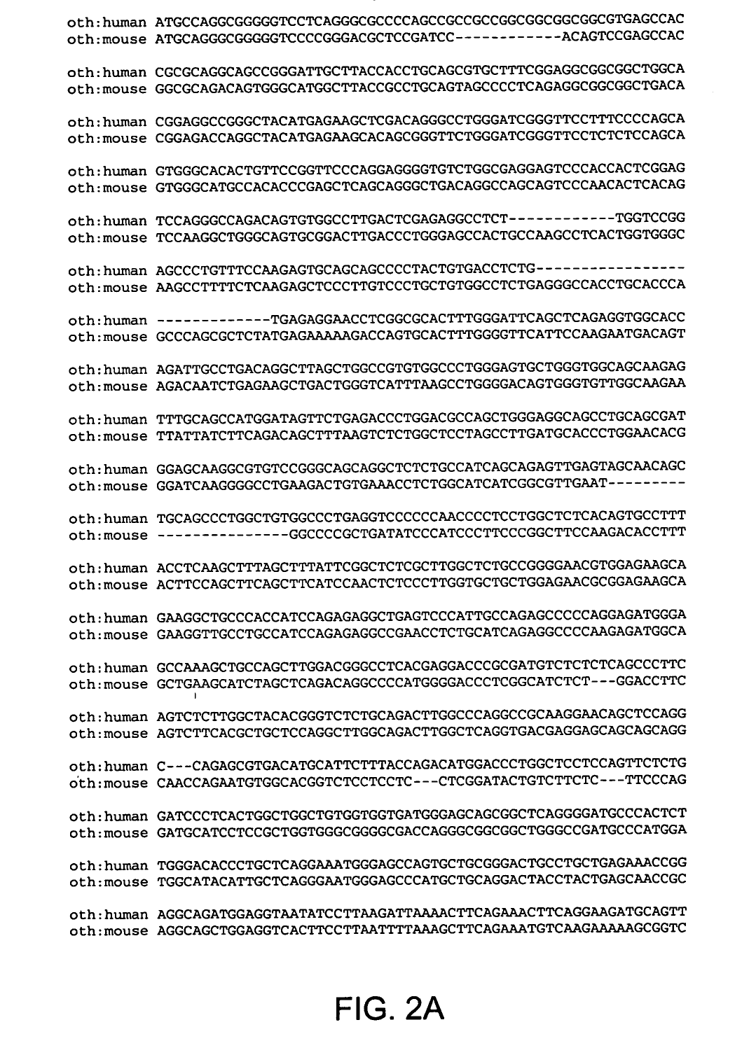

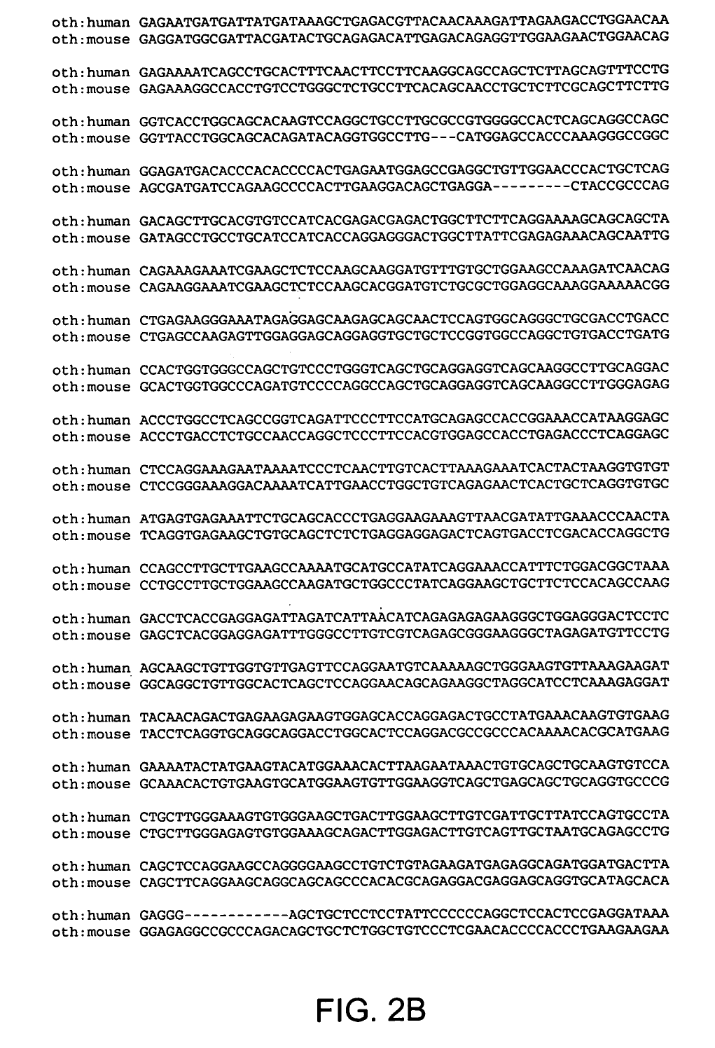

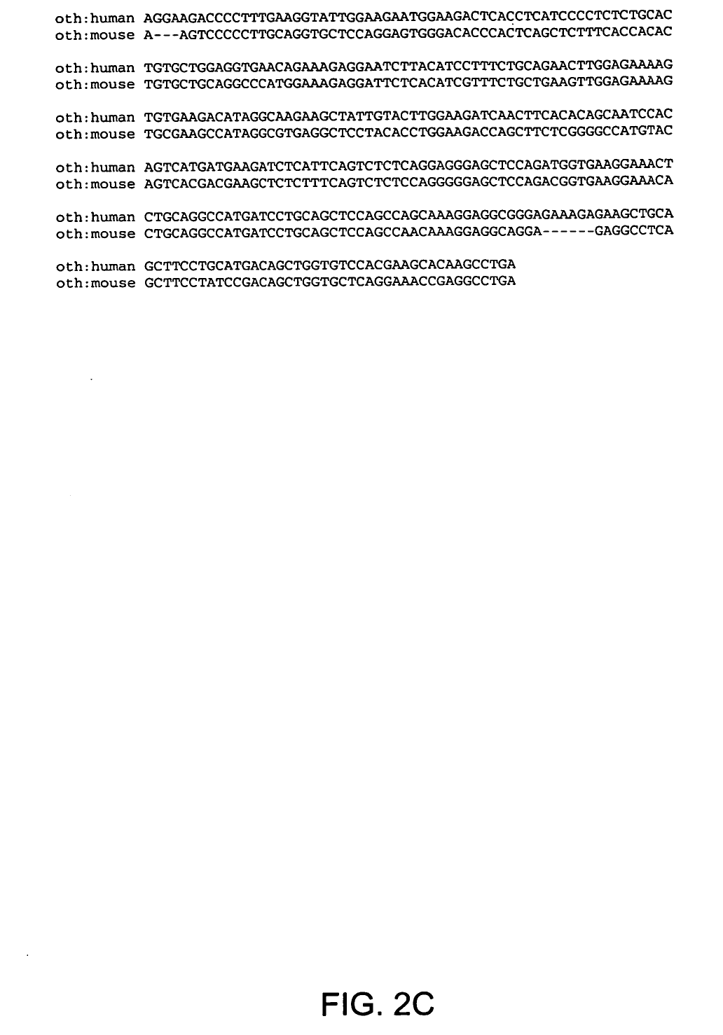

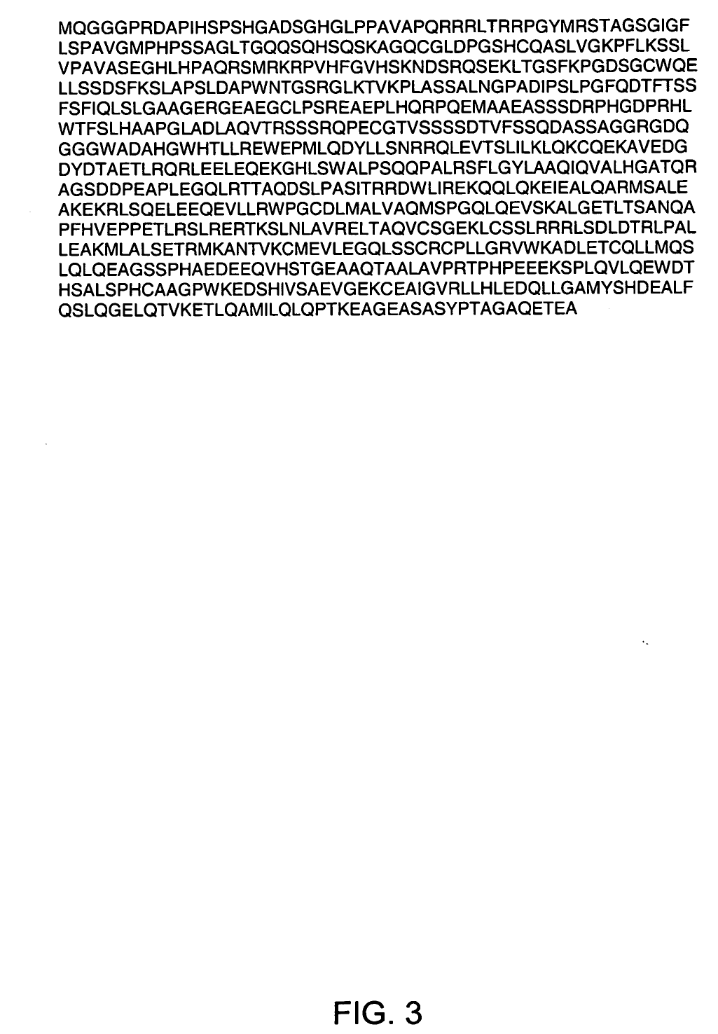

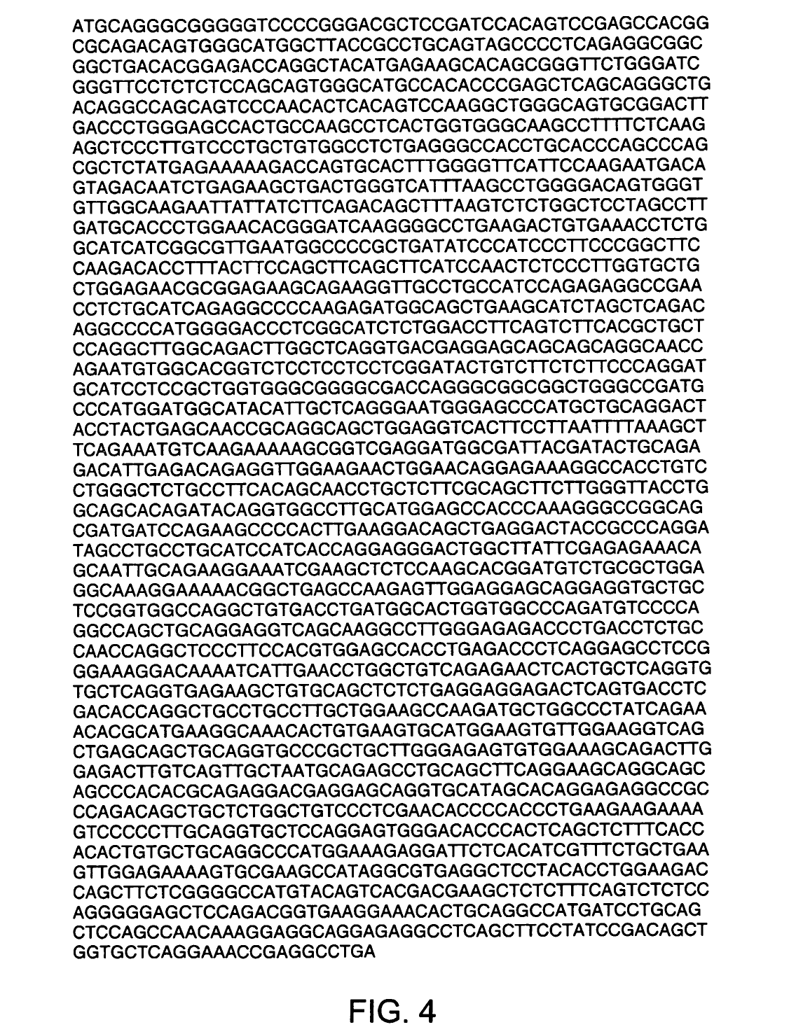

Clustal W (Thompson et al. (1994) Nucleic Acids Res, 22(22), 4673-4680) alignment of the human and murine DNA sequences revealed 60% identity between the sequences. Protein alignment between the human and mouse protein sequences (FIG. 1) demonstrated 56% identity and 14% similarity (excludes identical amino acids) between the protein sequences. This is a lower degree of homology than is typically seen between mouse and human orthologs. (Makalowski et al. (1996) Genome Res, 6(9), 846-857.)

Bioinformatic analysis using PROSFIE revealed that three leucine zipper motifs seen in the human DISC1 sequence are conserved in the mouse. Bioinformatic analysis techniques are described by Landschulz et al. (1988) Science, 240(4860), 1759-1764, Bairoch (1991) Nucleic Acids Res, 19(Suppl), 2241-2245, and Henikoff et al. (1991) Nucleic Acids Res, 19(23), 6565-6572. The leucine zipper motifs were located as follows: amino acids 454-475, amino acids 461-482 and amino acids 603-624 in Disc1 and amino acids 458-479, amino acids 465-486, and amino acids 607-628 in DISC1.

The potential coiled-coil domain in the C-terminal end of the human DISC1 protein previously described (Millar et al. (2000) Hum. Mol. Genet., 9(9), 1415-1423), is also conserved in the mouse protein. In addition, InterproScan database (Apweiler, et al. (2000) Bioinformatics, 16(12), 1145-1150) searching of the mouse sequence revealed a low homology to a putative prefolding chaperone, DUF23 (Mori et al. (1998) J. Biol. Chem, 273(45), 29794-29800). In contrast, neither the suggested bipartite nuclear localization signal (Dingwall et al. (1986) Annu. Rev. Cell. Biol., 2, 367-390), or the weak homology to tropomysoin (MacLeod (1987) 6(5), 208-212) found in human DISC1 were found in Disc1.

### Example 4

### Disc1 Chromosomal Localization

Due to the low homology level between the mouse Dis1 and human DISC1 sequence, mouse genomic sequence was examined to verify that it was the true ortholog of DISC1 by demonstrating that the cloned Disc1 gene sequence came from the syntenic region corresponding to human chromosome 1q42 in the mouse genome. TRAX (Translin-associated Factor X; Tsnax, NM_{—}016909) has been verified to be 35 kb (kilobase) proximal to the human DISC1 sequence on chromosome 1. (Millar et al. (2000) Genomics, 67(1), 69-77.)

Mouse BACs were identified by searching the TIGR mouse BAC end sequencing database with the mouse TRAX (Tsnax, NM_{—}016909) sequence (www.tigr.org). (Zhao et al. (2001). Genome Res, 11(10), 1736-1745.)

Two BACs were identified that contained Tsnax sequence. BAC 418L11 contained nucleotides 964-1446 and BAC 236F19 contained nucleotides 1500-2410 of Tsnax (FIG. 5). A BAC containing Disc1 sequence, 259E12, was also identified by hybridization of a Disc1 probe against an ES BAC library.

To confirm that Tsnax was located proximal to Disc1 in the mouse genome, PCR amplification using primers from Tsnax and Disc1 was performed on each of the identified BACs. 418L11 was positive for Tsnax DNA sequence for amino acids (aa) 733-983 whereas it was negative for Tsnax sequence 3′ using Tsnax DNA primers for aa1524-1660. 236F19 contains genomic mouse sequence distal to 418L11. PCR results demonstrated that it was negative for Tsnax sequence for aa1524-1660, but positive for Tsnax sequence aa2036-2258. In addition, 236F19 was positive for Disc1 sequence using primers for aa640-771 and aa828-1035. This result demonstrated that Disc1 was the true ortholog of DISC1 because it was in the mouse syntenic region corresponding to human chromosome 1q42.

### Example 5

### Northern Analysis

Disc1 probe was hybridized against a Clontech mouse multiple tissue northern blot. With low-stringency washing conditions, Disc1 transcripts were identified in heart, brain, kidney and testis. The heart had transcripts at 7.0 and 4.4 kb, testis at 10 and 4.4 kb and kidney had one transcript at 4.4 kb. A faint transcript was also identified in the brain at 7.0 kb. The Disc1 probe was also hybridized against a Clontech rat multiple tissue northern blot. With low-stringency washing conditions, Disc1 transcripts were identified in the heart, brain, liver, skeletal muscle, kidney and testis. Upon higher stringency washing, only the heart transcript at 7.0 kb was identified.

### Example 6

### In Situ Hybridization

In situ hybridization analysis was performed on adult mouse brain using a Disc1 riboprobe on C57BL6 mice brain sections. High level of expression was seen in the dentate gyrus of the hippocampus, with lower level expression in the olfactory bulbs, cerebellum, and CA1, CA2 and CA3 fields of the hippocampus.

Other embodiments are within the following claims. While several embodiments have been shown and described, various modifications may be made without departing from the spirit and scope of the present invention.

1. A purified polypeptide comprising at least 18 contiguous amino acids of SEQ ID NO: 1.

2. The polypeptide of claim 1, wherein said polypeptide comprises at least 50 contiguous amino acids of SEQ ID NO: 1.

3. The polypeptide of claim 1, wherein said polypeptide comprises at least 9 contiguous amino acids of two or more contiguous exon encoded regions selected from the group consisting of:
exon 1-exon 2;
exon 2-exon 3;
exon 3-exon 4;
exon 4-exon 5;
exon 5-exon 6;
exon 6-exon 7;
exon 7-exon 8;
exon 8-exon 9;
exon 9-exon 10;
exon 10-exon 11;
exon 11-exon 12; and
exon 12-exon 13.

4. The polypeptide of claim 1, wherein said polypeptide comprises the amino acid sequence of SEQ ID NO: 1, SEQ ID NO: 3, or a modified SEQ ID NO: 1, wherein said modified SEQ ID NO: 1 contains one or more modifications selected from the group consisting of:
amino acid 46: A to V;
amino acid 58: G to D;
amino acid 111: E to D;
amino acid 214: F to L; and
amino acid 231: C to R.

5. The polypeptide of claim 1, wherein said polypeptide consists of the amino acid sequence of SEQ ID NO: 1, SEQ ID NO: 3, or a modified SEQ ID NO: 1, wherein said modified SEQ ID NO: 1 contains one or more modifications selected from the group consisting of:
amino acid 46: A to V;
amino acid 58: G to D;
amino acid 111: E to D;
amino acid 214: F to L; and
amino acid 231: C to R.

6. The polypeptide of claim 1, wherein said polypeptide consists of the amino acid sequence of SEQ ID NO: 1.

7. A recombinant nucleic acid comprising a nucleotide sequence that either:
a) encodes the polypeptide of claim 1 and is transcriptionally coupled to an exogenous promoter;
b) is at least 30 contiguous bases present in SEQ ID NO: 2 or the complement thereof, and is attached to a solid support;
c) is SEQ ID NO: 2;
d) is a modified SEQ ID NO: 2, wherein said modified SEQ ID NO: 2 contains one or more modifications selected from the group consisting of:
nucleotide 137: C to T;
nucleotide 173: G to A;
nucleotide 333: G to T;
nucleotide 606: C to T;
nucleotide 640: T to C;
nucleotide 691: T to C; and
nucleotide 1191: G to A; and
e) is SEQ ID NO: 4.

8. The recombinant nucleic acid of claim 7, wherein said nucleotide sequence is either SEQ ID NO: 2, SEQ ID NO: 4, or is a modified SEQ ID NO: 2, wherein said modified SEQ ID NO: 2 contains one or more modifications selected from the group consisting of:
nucleotide 137: C to T;
nucleotide 173: G to A;
nucleotide 333: G to T;
nucleotide 606: C to T;
nucleotide 640: T to C;
nucleotide 691: T to C; and
nucleotide 1191: G to A; and
said nucleotide sequence is transcriptionally coupled to an exogenous promoter.

9. The recombinant nucleic acid of claim 8, wherein said recombinant nucleic acid is an expression vector.

10. A recombinant cell comprising the recombinant nucleic acid of claim 9, wherein said cell comprises an RNA polymerase recognized by said promoter.

11. (canceled)

12. A purified antibody preparation comprising an antibody that selectively binds to a polypeptide of SEQ ID NO: 1 over the human disrupted-in-schizophrenia 1 polypeptide.

13. A recombinant mouse comprising an alteration in an allele encoding a disrupted-in-schizophrenia 1 (Disc1) polypeptide comprising at least 20 contiguous amino acids of SEQ ID NO: 1, wherein said alteration substantially reduces, or increases, full length expression of said polypeptide from said allele.

14. The recombinant mouse of claim 13, wherein said Disc1 polypeptide consists of SEQ ID NO: 1, SEQ ID NO: 3, or a modified SEQ ID NO: 1, wherein said modified SEQ ID NO: 1 contains at least one modification selected from the group consisting of:
amino acid 46: A to V;
amino acid 58: G to D;
amino acid 111: E to D;
amino acid 214: F to L; and
amino acid 231: C to R.

15. The recombinant mouse of claim 13, wherein said alteration substantially eliminates expression of said polypeptide.

16. The recombinant mouse of claim 13, wherein said alteration results in the production of a truncated polypeptide.

17. The recombinant mouse of claim 13, wherein said mouse comprises alterations in both Disc1 alleles, wherein said alterations substantially reduce full-length expression of said polypeptide from said allele.

18-19. (canceled)


SEQUENCE LISTING (51 sequences)

SEQ ID NO: 1 - protein, 851 aa, Mouse
MQGGGPRDAPIHSPSHGADSGHGLPPAVAPQRRRLTRRPGYMRSTAGSGIGFLSPAVGMPHPSSAGLTGQQSQHSQSKAGQCGLDPGSHCQASLVGKPFLKSSLVPAVASEGHLHPAQRSMRKRPVHFGVHSKNDSRQSEKLTGSFKPGDSGCWQELLSSDSFKSLAPSLDAPWNTGSRGLKTVKPLASSALNGPADIPSLPGFQDTFTSSFSFIQLSLGAAGERGEAEGCLPSREAEPLHQRPQEMAAEASSSDRPHGDPRHLWTFSLHAAPGLADLAQVTRSSSRQPECGTVSSSSDTVFSSQDASSAGGRGDQGGGWADAHGWHTLLREWEPMLQDYLLSNRRQLEVTSLILKLQKCQEKAVEDGDYDTAETLRQRLEELEQEKGHLSWALPSQQPALRSFLGYLAAQIQVALHGATQRAGSDDPEAPLEGQLRTTAQDSLPASITRRDWLIREKQQLQKEIEALQARMSALEAKEKRLSQELEEQEVLLRWPGCDLMALVAQMSPGQLQEVSKALGETLTSANQAPFHVEPPETLRSLRERTKSLNLAVRELTAQVCSGEKLCSSLRRRLSDLDTRLPALLEAKMLALSGSCFSTAKELTEEIWALSSEREGLEMFLGRLLALSSRNSRRLGILKEDYLRCRQDLALQDAAHKTRMKANTVKCMEVLEGQLSSCRCPLLGRVWKADLETCQLLMQSLQLQEAGSSPHAEDEEQVHSTGEAAQTAALAVPRTPHPEEEKSPLQVLQEWDTHSALSPHCAAGPWKEDSHIVSAEVGEKCEAIGVRLLHLEDQLLGAMYSHDEALFQSLQGELQTVKETLQAMILQLQPTKEAGEASASYPTAGAQETEA

SEQ ID NO: 2 - DNA, 2556 bp, Artificial Sequence
  cDNA encoding mouse Disc1
atgcagggcgggggtccccgggacgctccgatccacagtccgagccacggcgcagacagtgggcatggcttaccgcctgcagtagcccctcagaggcggcggctgacacggagaccaggctacatgagaagcacagcgggttctgggatcgggttcctctctccagcagtgggcatgccacacccgagctcagcagggctgacaggccagcagtcccaacactcacagtccaaggctgggcagtgcggacttgaccctgggagccactgccaagcctcactggtgggcaagccttttctcaagagctcccttgtccctgctgtggcctctgagggccacctgcacccagcccagcgctctatgagaaaaagaccagtgcactttggggttcattccaagaatgacagtagacaatctgagaagctgactgggtcatttaagcctggggacagtgggtgttggcaagaattattatcttcagacagctttaagtctctggctcctagccttgatgcaccctggaacacgggatcaaggggcctgaagactgtgaaacctctggcatcatcggcgttgaatggccccgctgatatcccatcccttcccggcttccaagacacctttacttccagcttcagcttcatccaactctcccttggtgctgctggagaacgcggagaagcagaaggttgcctgccatccagagaggccgaacctctgcatcagaggccccaagagatggcagctgaagcatctagctcagacaggccccatggggaccctcggcatctctggaccttcagtcttcacgctgctccaggcttggcagacttggctcaggtgacgaggagcagcagcaggcaaccagaatgtggcacggtctcctcctcctcggatactgtcttctcttcccaggatgcatcctccgctggtgggcggggcgaccagggcggcggctgggccgatgcccatggatggcatacattgctcagggaatgggagcccatgctgcaggactacctactgagcaaccgcaggcagctggaggtcacttccttaattttaaagcttcagaaatgtcaagaaaaagcggtcgaggatggcgattacgatactgcagagacattgagacagaggttggaagaactggaacaggagaaaggccacctgtcctgggctctgccttcacagcaacctgctcttcgcagcttcttgggttacctggcagcacagatacaggtggccttgcatggagccacccaaagggccggcagcgatgatccagaagccccacttgaaggacagctgaggactaccgcccaggatagcctgcctgcatccatcaccaggagggactggcttattcgagagaaacagcaattgcagaaggaaatcgaagctctccaagcacggatgtctgcgctggaggcaaaggaaaaacggctgagccaagagttggaggagcaggaggtgctgctccggtggccaggctgtgacctgatggcactggtggcccagatgtccccaggccagctgcaggaggtcagcaaggccttgggagagaccctgacctctgccaaccaggctcccttccacgtggagccacctgagaccctcaggagcctccgggaaaggacaaaatcattgaacctggctgtcagagaactcactgctcaggtgtgctcaggtgagaagctgtgcagctctctgaggaggagactcagtgacctcgacaccaggctgcctgccttgctggaagccaagatgctggccctatcaggaagctgcttctccacagccaaggagctcacggaggagatttgggccttgtcgtcagagcgggaagggctagagatgttcctgggcaggctgttggcactcagctccaggaacagcagaaggctaggcatcctcaaagaggattacctcaggtgcaggcaggacctggcactccaggacgccgcccacaaaacacgcatgaaggcaaacactgtgaagtgcatggaagtgttggaaggtcagctgagcagctgcaggtgcccgctgcttgggagagtgtggaaagcagacttggagacttgtcagttgctaatgcagagcctgcagcttcaggaagcaggcagcagcccacacgcagaggacgaggagcaggtgcatagcacaggagaggccgcccagacagctgctctggctgtccctcgaacaccccaccctgaagaagaaaagtcccccttgcaggtgctccaggagtgggacacccactcagctctttcaccacactgtgctgcaggcccatggaaagaggattctcacatcgtttctgctgaagttggagaaaagtgcgaagccataggcgtgaggctcctacacctggaagaccagcttctcggggccatgtacagtcacgacgaagctctctttcagtctctccagggggagctccagacggtgaaggaaacactgcaggccatgatcctgcagctccagccaacaaaggaggcaggagaggcctcagcttcctatccgacagctggtgctcaggaaaccgaggcctga

SEQ ID NO: 3 - protein, 788 aa, Mouse
MQGGGPRDAPIHSPSHGADSGHGLPPAVAPQRRRLTRRPGYMRSTAGSGIGFLSPAVGMPHPSSAGLTGQQSQHSQSKAGQCGLDPGSHCQASLVGKPFLKSSLVPAVASEGHLHPAQRSMRKRPVHFGVHSKNDSRQSEKLTGSFKPGDSGCWQELLSSDSFKSLAPSLDAPWNTGSRGLKTVKPLASSALNGPADIPSLPGFQDTFTSSFSFIQLSLGAAGERGEAEGCLPSREAEPLHQRPQEMAAEASSSDRPHGDPRHLWTFSLHAAPGLADLAQVTRSSSRQPECGTVSSSSDTVFSSQDASSAGGRGDQGGGWADAHGWHTLLREWEPMLQDYLLSNRRQLEVTSLILKLQKCQEKAVEDGDYDTAETLRQRLEELEQEKGHLSWALPSQQPALRSFLGYLAAQIQVALHGATQRAGSDDPEAPLEGQLRTTAQDSLPASITRRDWLIREKQQLQKEIEALQARMSALEAKEKRLSQELEEQEVLLRWPGCDLMALVAQMSPGQLQEVSKALGETLTSANQAPFHVEPPETLRSLRERTKSLNLAVRELTAQVCSGEKLCSSLRRRLSDLDTRLPALLEAKMLALSETRMKANTVKCMEVLEGQLSSCRCPLLGRVWKADLETCQLLMQSLQLQEAGSSPHAEDEEQVHSTGEAAQTAALAVPRTPHPEEEKSPLQVLQEWDTHSALSPHCAAGPWKEDSHIVSAEVGEKCEAIGVRLLHLEDQLLGAMYSHDEALFQSLQGELQTVKETLQAMILQLQPTKEAGEASASYPTAGAQETEA

SEQ ID NO: 4 - DNA, 2367 bp, Artificial Sequence
  cDNA encoding Disc1 splice variant
atgcagggcgggggtccccgggacgctccgatccacagtccgagccacggcgcagacagtgggcatggcttaccgcctgcagtagcccctcagaggcggcggctgacacggagaccaggctacatgagaagcacagcgggttctgggatcgggttcctctctccagcagtgggcatgccacacccgagctcagcagggctgacaggccagcagtcccaacactcacagtccaaggctgggcagtgcggacttgaccctgggagccactgccaagcctcactggtgggcaagccttttctcaagagctcccttgtccctgctgtggcctctgagggccacctgcacccagcccagcgctctatgagaaaaagaccagtgcactttggggttcattccaagaatgacagtagacaatctgagaagctgactgggtcatttaagcctggggacagtgggtgttggcaagaattattatcttcagacagctttaagtctctggctcctagccttgatgcaccctggaacacgggatcaaggggcctgaagactgtgaaacctctggcatcatcggcgttgaatggccccgctgatatcccatcccttcccggcttccaagacacctttacttccagcttcagcttcatccaactctcccttggtgctgctggagaacgcggagaagcagaaggttgcctgccatccagagaggccgaacctctgcatcagaggccccaagagatggcagctgaagcatctagctcagacaggccccatggggaccctcggcatctctggaccttcagtcttcacgctgctccaggcttggcagacttggctcaggtgacgaggagcagcagcaggcaaccagaatgtggcacggtctcctcctcctcggatactgtcttctcttcccaggatgcatcctccgctggtgggcggggcgaccagggcggcggctgggccgatgcccatggatggcatacattgctcagggaatgggagcccatgctgcaggactacctactgagcaaccgcaggcagctggaggtcacttccttaattttaaagcttcagaaatgtcaagaaaaagcggtcgaggatggcgattacgatactgcagagacattgagacagaggttggaagaactggaacaggagaaaggccacctgtcctgggctctgccttcacagcaacctgctcttcgcagcttcttgggttacctggcagcacagatacaggtggccttgcatggagccacccaaagggccggcagcgatgatccagaagccccacttgaaggacagctgaggactaccgcccaggatagcctgcctgcatccatcaccaggagggactggcttattcgagagaaacagcaattgcagaaggaaatcgaagctctccaagcacggatgtctgcgctggaggcaaaggaaaaacggctgagccaagagttggaggagcaggaggtgctgctccggtggccaggctgtgacctgatggcactggtggcccagatgtccccaggccagctgcaggaggtcagcaaggccttgggagagaccctgacctctgccaaccaggctcccttccacgtggagccacctgagaccctcaggagcctccgggaaaggacaaaatcattgaacctggctgtcagagaactcactgctcaggtgtgctcaggtgagaagctgtgcagctctctgaggaggagactcagtgacctcgacaccaggctgcctgccttgctggaagccaagatgctggccctatcagaaacacgcatgaaggcaaacactgtgaagtgcatggaagtgttggaaggtcagctgagcagctgcaggtgcccgctgcttgggagagtgtggaaagcagacttggagacttgtcagttgctaatgcagagcctgcagcttcaggaagcaggcagcagcccacacgcagaggacgaggagcaggtgcatagcacaggagaggccgcccagacagctgctctggctgtccctcgaacaccccaccctgaagaagaaaagtcccccttgcaggtgctccaggagtgggacacccactcagctctttcaccacactgtgctgcaggcccatggaaagaggattctcacatcgtttctgctgaagttggagaaaagtgcgaagccataggcgtgaggctcctacacctggaagaccagcttctcggggccatgtacagtcacgacgaagctctctttcagtctctccagggggagctccagacggtgaaggaaacactgcaggccatgatcctgcagctccagccaacaaaggaggcaggagaggcctcagcttcctatccgacagctggtgctcaggaaaccgaggcctga

SEQ ID NO: 5 - protein, 854 aa, Human
MPGGGPQGAPAAAGGGGVSHRAGSRDCLPPAACFRRRRLARRPGYMRSSTGPGIGFLSPAVGTLFRFPGGVSGEESHHSESRARQCGLDSRGLLVRSPVSKSAAAPTVTSVRGTSAHFGIQLRGGTRLPDRLSWPCGPGSAGWQQEFAAMDSSETLDASWEAACSDGARRVRAAGSLPSAELSSNSCSPGCGPEVPPTPPGSHSAFTSSFSFIRLSLGSAGERGEAEGCPPSREAESHCQSPQEMGAKAASLDGPHEDPRCLSQPFSLLATRVSADLAQAARNSSRPERDMHSLPDMDPGSSSSLDPSLAGCGGDGSSGSGDAHSWDTLLRKWEPVLRDCLLRNRRQMEVISLRLKLQKLQEDAVENDDYDKAETLQQRLEDLEQEKISLHFQLPSRQPALSSFLGHLAAQVQAALRRGATQQASGDDTHTPLRMEPRLLEPTAQDSLHVSITRRDWLLQEKQQLQKEIEALQARMFVLEAKDQQLRREIEEQEQQLQWQGCDLTPLVGQLSLGQLQEVSKALQDTLASAGQIPFHAEPPETIRSLQERIKSLNLSLKEITTKVCMSEKFCSTLRKKVNDIETQLPALLEAKMHAISGNHFWTAKDLTEEIRSLTSEREGLEGLLSKLLVLSSRNVKKLGSVKEDYNRLRREVEHQETAYETSVKENTMKYMETLKNKLCSCKCPLLGKVWEADLEACRLLIQCLQLQEARGSLSVEDERQMDDLEGAAPPIPPRLHSEDKRKTPLKVLEEWKTHLIPSLHCAGGEQKEESYILSAELGEKCEDIGKKLLYLEDQLHTAIHSHDEDLIQSLRRELQMVKETLQAMILQLQPAKEAGEREAAASCMTAGVHEAQA

SEQ ID NO: 6 - DNA, 2565 bp, Artificial Sequence
  cDNA encoding human Disc1
atgccaggcgggggtcctcagggcgccccagccgccgccggcggcggcggcgtgagccaccgcgcaggcagccgggattgcttaccacctgcagcgtgctttcggaggcggcggctggcacggaggccgggctacatgagaagctcgacagggcctgggatcgggttcctttccccagcagtgggcacactgttccggttcccaggaggggtgtctggcgaggagtcccaccactcggagtccagggccagacagtgtggccttgactcgagaggcctcttggtccggagccctgtttccaagagtgcagcagcccctactgtgacctctgtgagaggaacctcggcgcactttgggattcagctcagaggtggcaccagattgcctgacaggcttagctggccgtgtggccctgggagtgctgggtggcagcaagagtttgcagccatggatagttctgagaccctggacgccagctgggaggcagcctgcagcgatggagcaaggcgtgtccgggcagcaggctctctgccatcagcagagttgagtagcaacagctgcagccctggctgtggccctgaggtccccccaacccctcctggctctcacagtgcctttacctcaagctttagctttattcggctctcgcttggctctgccggggaacgtggagaagcagaaggctgcccaccatccagagaggctgagtcccattgccagagcccccaggagatgggagccaaagctgccagcttggacgggcctcacgaggacccgcgatgtctctctcagcccttcagtctcttggctacacgggtctctgcagacttggcccaggccgcaaggaacagctccaggccagagcgtgacatgcattctttaccagacatggaccctggctcctccagttctctggatccctcactggctggctgtggtggtgatgggagcagcggctcaggggatgcccactcttgggacaccctgctcaggaaatgggagccagtgctgcgggactgcctgctgagaaaccggaggcagatggaggtaatatccttaagattaaaacttcagaaacttcaggaagatgcagttgagaatgatgattatgataaagctgagacgttacaacaaagattagaagacctggaacaagagaaaatcagcctgcactttcaacttccttcaaggcagccagctcttagcagtttcctgggtcacctggcagcacaagtccaggctgccttgcgccgtggggccactcagcaggccagcggagatgacacccacaccccactgagaatggagccgaggctgttggaacccactgctcaggacagcttgcacgtgtccatcacgagacgagactggcttcttcaggaaaagcagcagctacagaaagaaatcgaagctctccaagcaaggatgtttgtgctggaagccaaagatcaacagctgagaagggaaatagaggagcaagagcagcaactccagtggcagggctgcgacctgaccccactggtgggccagctgtccctgggtcagctgcaggaggtcagcaaggccttgcaggacaccctggcctcagccggtcagattcccttccatgcagagccaccggaaaccataaggagcctccaggaaagaataaaatccctcaacttgtcacttaaagaaatcactactaaggtgtgtatgagtgagaaattctgcagcaccctgaggaagaaagttaacgatattgaaacccaactaccagccttgcttgaagccaaaatgcatgccatatcaggaaaccatttctggacggctaaagacctcaccgaggagattagatcattaacatcagagagagaagggctggagggactcctcagcaagctgttggtgttgagttccaggaatgtcaaaaagctgggaagtgttaaagaagattacaacagactgagaagagaagtggagcaccaggagactgcctatgaaacaagtgtgaaggaaaatactatgaagtacatggaaacacttaagaataaactgtgcagctgcaagtgtccactgcttgggaaagtgtgggaagctgacttggaagcttgtcgattgcttatccagtgcctacagctccaggaagccaggggaagcctgtctgtagaagatgagaggcagatggatgacttagagggagctgctcctcctattccccccaggctccactccgaggataaaaggaagacccctttgaaggtattggaagaatggaagactcacctcatcccctctctgcactgtgctggaggtgaacagaaagaggaatcttacatcctttctgcagaacttggagaaaagtgtgaagacataggcaagaagctattgtacttggaagatcaacttcacacagcaatccacagtcatgatgaagatctcattcagtctctcaggagggagctccagatggtgaaggaaactctgcaggccatgatcctgcagctccagccagcaaaggaggcgggagaaagagaagctgcagcttcctgcatgacagctggtgtccacgaagcacaagcctga

SEQ ID NO: 7 - DNA, 25 bp, Artificial Sequence
  Splice Acceptor Site
acactgttttctcttctcttctcag

SEQ ID NO: 8 - DNA, 25 bp, Artificial Sequence
  Splice Acceptor Site
atgtttccctttctcacccacacag

SEQ ID NO: 9 - DNA, 25 bp, Artificial Sequence
  Splice Acceptor Site
tgcttttacctctttgggtttccag

SEQ ID NO: 10 - DNA, 25 bp, Artificial Sequence
  Splice Acceptor Site
accaatgcatgtctgttacttgaag

SEQ ID NO: 11 - DNA, 25 bp, Artificial Sequence
  Splice Acceptor Site
atctgttccccctctctctctgcag

SEQ ID NO: 12 - DNA, 25 bp, Artificial Sequence
  Splice Acceptor Site
caatgctcctttctaatttctctag

SEQ ID NO: 13 - DNA, 25 bp, Artificial Sequence
  Splice Acceptor Site
ttgattctgccgtttctcctggcag

SEQ ID NO: 14 - DNA, 25 bp, Artificial Sequence
  Splice Acceptor Site
tcctctctcccccactgtgttgcag

SEQ ID NO: 15 - DNA, 25 bp, Artificial Sequence
  Splice Acceptor Site
tgctcacgttgggtttttcttgcag

SEQ ID NO: 16 - DNA, 25 bp, Artificial Sequence
  Splice Acceptor Site
ccatgcctgccttcctctgtcgtag

SEQ ID NO: 17 - DNA, 25 bp, Artificial Sequence
  Splice Acceptor Site
gacacatctctcattctctgaccag

SEQ ID NO: 18 - DNA, 25 bp, Artificial Sequence
  Splice Acceptor Site
ttgtgtgctccttaacaatgtctac

SEQ ID NO: 19 - DNA, 25 bp, Artificial Sequence
  Splice Acceptor Site
ttttctttctttctttttccttcag

SEQ ID NO: 20 - DNA, 24 bp, Artificial Sequence
  Splice Acceptor Site
tgtcgccgccgccaccaccaccac

SEQ ID NO: 21 - DNA, 25 bp, Artificial Sequence
  Splice Doner Site
gtagggcccggggttctggaggagg

SEQ ID NO: 22 - DNA, 25 bp, Artificial Sequence
  Splice Doner Site
gtgtgtgtgcttctggaatcgggtc

SEQ ID NO: 23 - DNA, 25 bp, Artificial Sequence
  Splice Doner Site
gtgagtccaaagctgttcgtagaca

SEQ ID NO: 24 - DNA, 25 bp, Artificial Sequence
  Splice Doner Site
gtgagtacccgtggatgccaccaca

SEQ ID NO: 25 - DNA, 25 bp, Artificial Sequence
  Splice Doner Site
gtgagtggaatagaatcttccagaa

SEQ ID NO: 26 - DNA, 25 bp, Artificial Sequence
  Splice Doner Site
gtactggtgactttctgagtttcca

SEQ ID NO: 27 - DNA, 25 bp, Artificial Sequence
  Splice Doner Site
gtaagcccaccctcctcccattttc

SEQ ID NO: 28 - DNA, 25 bp, Artificial Sequence
  Splice Doner Site
gtaactgcagaggcacttatattca

SEQ ID NO: 29 - DNA, 25 bp, Artificial Sequence
  Splice Doner Site
gtgagtagcccccagccaaagcctc

SEQ ID NO: 30 - DNA, 25 bp, Artificial Sequence
  Splice Doner Site
gtaagttgtgtgtgtgtgtgggggg

SEQ ID NO: 31 - DNA, 25 bp, Artificial Sequence
  Splice Doner Site
gtttgtcctgtgtgtatggctttgt

SEQ ID NO: 32 - DNA, 25 bp, Artificial Sequence
  Splice Doner Site
atatccttttcagtctctcgggaat

SEQ ID NO: 33 - DNA, 25 bp, Artificial Sequence
  Splice Doner Site
gtgagtgtggagggggacgggggag

SEQ ID NO: 34 - DNA, 24 bp, Artificial Sequence
  Splice Doner Site
tgtcgccgccgccaccaccaccac

SEQ ID NO: 35 - DNA, 20 bp, Artificial Sequence
  Primer
ttcatccaactctcccttgg

SEQ ID NO: 36 - DNA, 19 bp, Artificial Sequence
  Primer
gagagcttcgtcgtgactg

SEQ ID NO: 37 - DNA, 22 bp, Artificial Sequence
  Primer
cattctggttgcctgctgctgc

SEQ ID NO: 38 - DNA, 22 bp, Artificial Sequence
  Primer
acctgagccaagtctgccaagc

SEQ ID NO: 39 - DNA, 24 bp, Artificial Sequence
  Primer
ctgctgaagttggagaaaagtgcg

SEQ ID NO: 40 - DNA, 23 bp, Artificial Sequence
  Primer
ggccatgtacagtcacgacgaag

SEQ ID NO: 41 - DNA, 23 bp, Artificial Sequence
  Primer
gagctccagacggtgaaggaaac

SEQ ID NO: 42 - DNA, 21 bp, Artificial Sequence
  Primer
ggattctcacatcgtttctgc

SEQ ID NO: 43 - DNA, 19 bp, Artificial Sequence
  Primer
gagagcttcgtcgtgactg

SEQ ID NO: 44 - DNA, 20 bp, Artificial Sequence
  Primer
gaaatggccactatacctgc

SEQ ID NO: 45 - DNA, 18 bp, Artificial Sequence
  Primer
cggcagcagtggttgtga

SEQ ID NO: 46 - DNA, 20 bp, Artificial Sequence
  Primer
ccacatgctttcaacgagtt

SEQ ID NO: 47 - DNA, 21 bp, Artificial Sequence
  Primer
agagcaggtaccaggactgac

SEQ ID NO: 48 - DNA, 20 bp, Artificial Sequence
  Primer
ttcatccaactctcccttgg

SEQ ID NO: 49 - DNA, 20 bp, Artificial Sequence
  Primer
gggcctgtctgagctagatg

SEQ ID NO: 50 - DNA, 20 bp, Artificial Sequence
  Primer
agacttggctcaggtgacga

SEQ ID NO: 51 - DNA, 19 bp, Artificial Sequence
  Primer
gcggttgctcagtaggtag